(19) United States
(12) Patent Application Publication
(10) Pub. No.: US 2021/0283191 A1
(43) Pub. Date: Sep. 16, 2021

(54) FOLLICULAR FLUID

(71) Applicant: GIONAS S.R.L., Milano (IT)

(72) Inventor: Cristina MAGGIONI, Milano (IT)

(73) Assignee: GIONAS S.R.L., Milano (IT)

(21) Appl. No.: 17/332,268

(22) Filed: May 27, 2021

Related U.S. Application Data
(62) Division of application No. 16/099,614, filed on Nov. 7, 2018.

(30) Foreign Application Priority Data
May 11, 2016 (IT) 102016000048332

(51) Int. Cl. A61K 35/54; A61K 38/24; A61K 35/00; A61P 15/08; A61K 9/00

(52) U.S. Cl. CPC A61K 35/54; A61K 38/24; A61K 45/06; A61P 15/08; A61K 9/0034; A61K 35/00

(57) ABSTRACT

Follicular fluid is used in in vitro fertilization. The use includes introducing follicular fluid in the uterus in one or more administrations during a time period of between six days before embryo transfer and the transfer day.

This application is a Divisional of U.S. patent application Ser. No. 16/099,614, filed 7 Nov. 2018, which is a National Stage Application of PCT/IB2017/052661, filed 8 May 2017, which claims the benefit of Ser. No. 10/216,000048332, filed 11 May 2016 in Italy, and which applications are incorporated herein by reference. To the extent appropriate, a claim of priority is made to each of the above-disclosed applications.

The present invention relates to follicular fluid for use in vitro fertilization, where said use comprises introducing follicular fluid in the uterus in one or more administrations during a time period comprised between six days before embryo transfer and the transfer day itself.

### PRIOR ART

Among the assisted reproduction techniques, there is IVF-ICSI, which involves in vitro fertilization of the egg with a subsequent transfer of the embryo thus formed in the woman's uterus.

The procedure is essentially divided into four steps: controlled induction of ovulation; oocyte retrieval; in vitro fertilization; embryo transfer. Depending on whether in vitro fertilization takes place naturally or by intra cytoplasmic injection of gametes, the techniques are called IVF or ICSI, respectively.

Since the first IVF success in the late 70's to the present, the success rate of the technique has had no major improvements. The step of re-implantation of embryos in the uterus is the most important step, which could significantly influence the outcome of an IVF cycle.

The same author of the present document, in Martel D. et al. J Endocrinol. 1987 114(2):319-24, noted that the moment in which the implantation of embryos occurs is, in humans, the fifth day after ovulation. However, in practice, the transfer of the blastocyst continues to be made on the second day after ovulation, unless material, oocytes or embryos, is used which has been stored from previous fertilizations.

The injection into the uterus before the blastocyst transfer of the supernatant of embryonic cultures has not improved the success rates of the implantation (Zhu W. et al. Fertil Steril. 2010 93(7):2216-21; Goto S. et al. Fertil Steril. 2009 92(4):1264-8).

On average, up to 90% of apparently healthy zygotes are destined to disappear when implanted in the uterus, without giving any sign of trophoblastic implantation and of production of human chorionic gonadotropin (hCG).

Since the different steps of IVF-ICSI are particularly challenging for women, both in physical and psychological terms, the need to increase the still disappointing success rates thereof is strongly felt. The implantation step is now the critical step on which to intervene with the aim of increasing the success rate of IVF-ICSI.

Follicular fluid is a combined product of the transfer of blood constituents that cross the follicular barrier and of the secretory activity of the granulose and cellular cells (Fortune J. E. Biol Reprod. 1994, 50: 225-232). The oocyte that is cyclically formed in the female reproductive system, either physiologically or typically in the context of assisted procreation procedures, following hormone induction, has a nucleus inside around which a single layer of flat cells is arranged, follicular cells. With the passing of days, the flat follicular cells become cubic and multiply, thus forming multiple concentric layers, until a space containing the follicular fluid is formed between them. As the maturation process progresses, the cortical layer surrounding the follicle proliferates, thus originating the theca interna and the theca externa. At full maturity, the theca interna disappears and the follicle, which protrudes at the surface of the ovary, breaks down. This rupture is accompanied by the escape of the follicular fluid and the expulsion of the mature oocyte.

The author of the present invention proposes an implantation method that has been shown to improve the success rate of IVF-ICSI, due to the improvements that the method surprisingly achieves in the critical implantation step.

### SUMMARY OF THE INVENTION

The author of the present invention has surprisingly demonstrated that zygote implantations in the uterus have a greater implantation efficacy when said implantation is preceded by or occurs in conjunction with the introduction of follicular fluid in the uterus, wherein said follicular fluid is autologous follicular fluid, collected when picking the egg from the subject herself, or it is follicular fluid collected at the time of the oocyte explantation and stored frozen until the time of introduction into the uterus.

FIG. 1 is a block diagram of the method according to the present invention.

An innovative method to be applied in the step of embryo transfer in the uterus is described herein, where said method significantly increases the percentage of implantation in the uterus, described in detail in the following paragraphs and claims.

For the purposes of the present invention, the follicular fluid is obtained from the ovaries by any of the methods known to those skilled in the art. Typically, in an IVF or ICSI procedure, follicular fluid is readily available as it is aspirated along with the oocyte during the oocyte collection step.

For the purposes of the present invention, follicular fluid is a follicular fluid that has the characteristics of the follicular fluid just aspirated from the ovaries. In one embodiment, it is follicular fluid just explanted from the ovaries. Alternatively, it is follicular fluid kept frozen, preferably at −80° C., or in liquid nitrogen, from the aspiration to the time of use. Said follicular fluid is therefore a follicular fluid that comprises all components of the freshly aspirated follicular fluid unaltered, which does not undergo dilutions with other media or degradation due to inadequate storage conditions.

### BRIEF DESCRIPTION OF THE DRAWINGS

FIG. 1 is a block diagram representing the innovative method described herein.

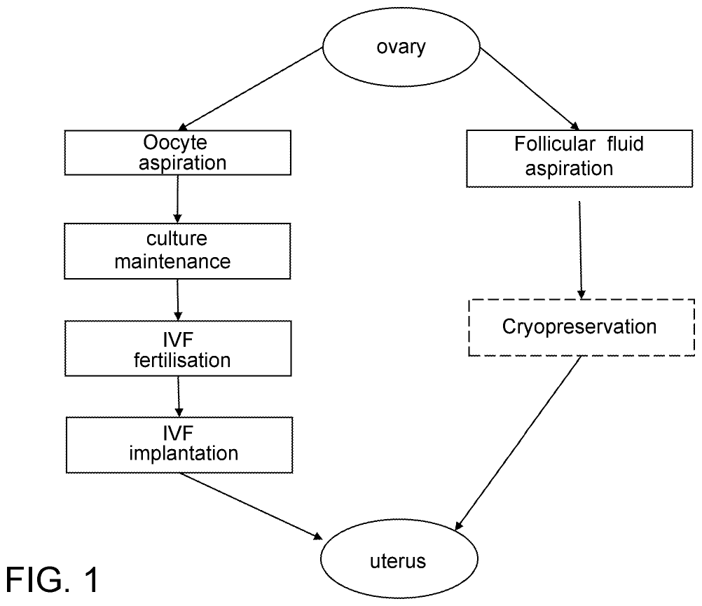

### DETAILED DESCRIPTION OF THE PREFERRED EMBODIMENT

Said follicular fluid is aspirated when the oocytes that will be subjected to vitro fertilization are aspirated, or it is aspirated at a different time. Typically, it is aspirated along with the oocytes. The oocytes are then isolated by aspiration from the follicular fluid, after which said follicular fluid is introduced into the uterus or stored for subsequent use. To this end, said follicular fluid is stored for the introduction into the uterus at a later time. In both cases, follicular fluid is introduced into the uterus according to the definition given for the purposes of the present invention.

For the purposes of the present invention, the follicular fluid is preferably collected from the follicles in phase with the complete maturation of the oocyte, i.e. in the form in which they are found at the time of the fine needle aspiration.

Said follicular fluid is introduced into the uterus using the procedures typically used for embryonic transfer. The day of introduction of said follicular fluid into the uterus is particularly critical for the success of the treatment. In a preferred embodiment, said follicular fluid is introduced into the uterus immediately after the collection and processing thereof, where processing involves the isolation of the oocytes contained therein. Preferably, 5 days after the introduction of the follicular fluid into the uterus, embryo transfer is carried out. In another embodiment, embryo transfer is carried out after four days, or after three days, or after 1 day of introduction of follicular fluid into the uterus. In an alternative embodiment, said follicular fluid is introduced into the uterus at the same time as the embryo transfer.

The author of the present invention has surprisingly demonstrated that follicular fluid, introduced into the uterus, has an advantageous role in promoting the successful outcome of the embryo implantation. This effect is observed where said follicular fluid has all the characteristics of the freshly explanted follicular fluid. In a preferred embodiment, said follicular fluid is introduced into the uterus in a volume comprised between 0.1 and 5 cm³, more preferably between 1.5 and 3 cm³, even more preferably in a volume of about 2 cm³.

Surprisingly, the authors of the present invention have shown that follicular fluid plays a crucial role in determining the temporal window of the endometrial implantation, where the presence of fresh follicular fluid, i.e. having the characteristics of the freshly aspirated follicular fluid, makes the maturation of the endometrium reach the optimum maturation stage for embryo implantation. This effect is obtained even more advantageously when the introduction of fresh follicular fluid into the uterus precedes the introduction of the fertilized embryo into the uterus by one, two, three, four, five, six days. The maximum effect is achieved by introducing fresh follicular fluid into the uterus from 2 to 4 days before the introduction of the fertilized embryo (embryo transfer, ET), even more preferably three days before.

For the purpose of the present invention, it is essential that said follicular fluid is properly stored follicular fluid, because after a few hours of aspiration, for example if maintained at room temperature, said follicular fluid undergoes a degradation of some of its fundamental components, a degradation which causes the follicular fluid to lose its surprising capacity shown herein to bring the endometrium to maturation. In a preferred embodiment, said fresh follicular fluid is not diluted with other culture media.

Follicular fluid is claimed herein for use in the treatment of infertility, characterized in that said follicular fluid is introduced into the uterus in one or more administrations during a time period comprised between six days before embryo transfer and the transfer day itself.

In a preferred embodiment, said use is in the embryo transfer process.

In a preferred embodiment, said use is in improving the implantation success rate in a human by transfer of blastocysts, where follicular fluid is introduced into the uterine cavity of a human patient undergoing embryo transfer in one or more administrations during a time period comprised between six days before the embryo transfer and the day of the transfer itself.

In a further aspect, a pharmaceutical composition is described, comprising fresh follicular fluid for therapeutic use in the IVF-ICSI treatment and other extra-uterine fertilization procedures, in a volume comprised between 0.1 and 5 cm³, more preferably between 1.5 and 3 cm³, even more preferably about 2 cm³. Said composition is introduced into the uterus one, two, three, four, five or six days before the embryo transfer.

A method for producing a composition for use in improving the implantation success rate by transfer of blastocysts is also claimed, said method comprising the steps of collecting follicular fluid from the same subject in which said blastocyst transfer will be carried out, optionally storing it at −80° C. or in liquid nitrogen, optionally admixing it with other active components. Said follicular fluid is preferably collected from follicles of at least 16 mm, or at least 20 mm.

A further object of the present invention is the follicular fluid for use in vitro fertilization, where said use comprises introducing follicular fluid in the uterus in one or more administrations during a time period comprised between six days before embryo transfer and the transfer day itself.

In a preferred embodiment, said follicular fluid is collected from mature follicles, preferably of at least 16 mm.

Even more preferably, said follicular fluid is autologous follicular fluid.

A further aspect of the present invention is a method for collecting and processing follicular fluid, comprising:
collecting, preferably transvaginally, ovarian follicles from the ovary, by employing ultrasound techniques;
puncturing the ovarian follicles and collecting the oocyte-containing follicular fluid;
isolating the oocytes through aspiration;
introducing the follicular fluid into the uterus or storing it.

A further aspect of the present invention is the combined intrauterine administration of a composition comprising the follicular fluid, or fractions thereof, together with one or more pharmacological agents, for example selected from progesterone, nitric oxide donors, aromatase inhibitors, ascorbic acid, glucocorticoids, insulin or insulin-sensitizing drugs, relaxin, HCG, thyroxine, melatonin, phytotherapeutic extracts, verbascum. The selection of said one or more pharmacological agents is carried out on the basis of specific patient's needs, as is well known to the man skilled in the art.

Alternatively, said introduction of the follicular fluid into the uterus is accompanied by the administration of one or more pharmacological agents that favor the uterine implantation. Said one or more pharmacological agents, selected from the group comprising, for example, progesterone, nitric oxide donors, aromatase inhibitors, ascorbic acid, glucocorticoids, insulin or insulin-sensitizing drugs, relaxin, HCG, thyroxine, melatonin, phytotherapeutic extracts, verbascum, is administered by an administration route selected, for example, from subcutaneously, intraperitoneally, intramuscularly, sublingually, orally. Said one or more agents are administered in one or more administrations within a time period comprised between fourteen days before the introduction of the follicular fluid into the uterus and up to 3 months after the introduction of the follicular fluid, or up to one month later, or up to fifteen days later, or up to five days later.

A further object of the present invention is a pharmaceutical composition comprising: follicular fluid according to the definition given in the present invention, one or more pharmacological agents selected from the group comprising, for example, progesterone, nitric oxide donors, aromatase inhibitors, ascorbic acid, glucocorticoids, insulin or insulin-sensitizing drugs, relaxin, HCG, thyroxine, melatonin, phytotherapeutic extracts, verbascum, one or more pharmaceutically acceptable carriers.

Said composition finds application for use in in vitro fertilization, where said use comprises introducing said composition in the uterus in one or more administrations during a time period comprised between six days before embryo transfer and the transfer day itself.

In a further aspect, a method for IVF-ICSI and other extra-uterine fertilization procedures is described herein, wherein said method comprises administering fresh autologous follicular fluid to a woman in one or more administrations, wherein said follicular fluid is introduced into the uterus in a volume of between 1 and 5 cm³, more preferably between 1.5 and 3 cm³, in a time period comprised between 6 days before the embryo transfer and the day of the transfer itself. Said method has surprisingly been shown to improve the implantation success rate.

Said method, described herein for humans, is advantageously applied also in other mammalian species.

1-13. (canceled)

14. A method of using fresh follicular fluid for in vitro fertilizations, said method comprising withdrawing fresh follicular fluid from a plurality of follicles, introducing 0.1 cm³ to 5 cm³ of fresh follicular fluid in a uterus in one or more administrations during a time period between four days and six days before embryo transfer.

15. The method of using fresh follicular fluid according to claim 14, comprising withdrawing fresh follicular fluid just aspirated or properly stored after aspiration, at −80° C. or in liquid nitrogen.

16. The method of using fresh follicular fluid according to claim 14, wherein said method comprises improving the blastocyst implantation success rate.

17. The method of using follicular fluid according to claim 15, wherein said use is in improving the blastocyst implantation success rate.

18. The method of using follicular fluid according to claim 14, wherein said follicular fluid is autologous follicular fluid.

19. The method of using fresh follicular fluid according to claim 14, comprising collecting said fresh follicular fluid from mature follicles.

20. The method of using fresh follicular fluid according to claim 14, further comprising introducing one or more pharmacological agents selected from the group consisting of progesterone, nitric oxide donors, aromatase inhibitors, ascorbic acid, glucocorticoids, insulin or insulin-sensitizing drugs, relaxin, HCG, thyroxine, melatonin, phytotherapeutic extracts, verbascum, and one or more pharmaceutically acceptable carriers.

21. The method of using fresh follicular fluid according to claim 20, comprising administering said one or more agents by introduction into the uterus and simultaneously introducing the follicular fluid into the uterus, or are administered in one or more administrations within a time period comprised between fourteen days before introduction of the follicular fluid and up to 3 months after the introduction of the follicular fluid, by an administration route selected from, subcutaneously, intraperitoneally, intramuscularly, sublingually, orally.
